(19) United States
(12) Patent Application Publication
(10) Pub. No.: US 2025/0186260 A1
(43) Pub. Date: Jun. 12, 2025

(54) PROTECTIVE EYEWEAR FOR HIGH INTENSITY SOURCES

(71) Applicants: Laura R. Vanderhoef, West Grove, PA (US); Jeffrey L. Cameron, New Castle, DE (US); Aaron M. Schweinsberg, Aberdeen, MD (US); Colby T. Adams, Havre de Grace, MD (US); Patrick M. Marine, Huntsville, AL (US); Thomas W. Kottke, Rising Sun, MD (US); George M. Thomson, Churchville, MD (US)

(72) Inventors: Laura R. Vanderhoef, West Grove, PA (US); Jeffrey L. Cameron, New Castle, DE (US); Aaron M. Schweinsberg, Aberdeen, MD (US); Colby T. Adams, Havre de Grace, MD (US); Patrick M. Marine, Huntsville, AL (US); Thomas W. Kottke, Rising Sun, MD (US); George M. Thomson, Churchville, MD (US)

(21) Appl. No.: 18/536,504

(22) Filed: Dec. 12, 2023

(51) Int. Cl. A61F 9/02

(52) U.S. Cl. CPC A61F 9/023; A61F 9/025

(57) ABSTRACT

Protective eyewear that may provide a full field of view while shielding users from unsafe, harmful, or unwanted visual elements, including high-intensity sources such as lasers, is described. Such shielding may include, for instance, capturing, attenuating, transforming, filtering, and/or otherwise processing such unsafe, harmful, or unwanted visual elements for presentation to users. The protective eyewear may enhance visual elements, such as by increasing brightness or contrast. The protective eyewear may allow users to see visual elements outside their normal or expected range such as ultraviolet and infrared. The protective eyewear may include one or more cameras that may capture visual information associated with the user environment. The captured information may be analyzed and processed such that harmful or unwanted visual elements are removed and/or otherwise processed to be made safe for viewing. After processing, the captured information may be rendered and provided via a display of the protective eyewear.

### GOVERNMENT INTEREST

The invention described herein may be manufactured, used and licensed by or for the U.S. Government.

### FIELD OF THE INVENTION

The invention is related to protective eyewear for use with lasers and other high-intensity sources.

### BACKGROUND OF THE INVENTION

Current solutions usually operate in two possible modes. For threat devices that produce a flux at a discrete wavelength, such as most lasers, protection requires the user to don eyewear that removes light at that specific wavelength while transmitting others. The user must maintain an inventory of eyewear for each specific wavelength employed. Alternative threat devices, such as arc lamps, the sun, and supercontinuum lasers, generate light at all wavelengths. These require protective flux attenuation over a broad wavelength span such as found in sunglasses or welding goggles. Such attenuation makes the surrounding workplace appear dimly illuminated at best. It is unsafe for users to move about in such an environment and be able to properly operate equipment, utilize tools, etc.

Therefore, there exists a need for eyewear that provides protection at all wavelengths of visible light, renders the workplace scene to appear normally lit, while shielding users from dangerous or unwanted visual elements.

### BRIEF SUMMARY OF THE INVENTION

Protective eyewear of some embodiments may provide a full field of view while shielding users from unsafe, harmful, or unwanted visual elements, including high-intensity sources such as lasers. Such shielding may include, capturing the incident radiation and electro-optically transforming, filtering, and/or otherwise processing it such that unsafe, harmful, or unwanted visual elements are, not inflicted upon users while presenting a faithful visual representation of the scene.

In some embodiments, the protective eyewear may enhance visual elements, such as by increasing brightness, contrast, etc. The protective eyewear of some embodiments may allow users to see visual elements outside their normal or expected range (e.g., infrared emitting objects moving at high speeds, etc.).

The protective eyewear of some embodiments may include one or more cameras and/or other optical sensors or components. The cameras may capture visual information associated with the user environment. The captured information may be analyzed and processed such that unsafe, harmful, or unwanted visual elements are removed and/or otherwise processed to be made safe for viewing. After processing, the captured information may be rendered for display, where the rendered media may be referred to as a “sanitized” visual environment.

The protective eyewear of some embodiments may include a set of display components that may provide the sanitized visual environment by displaying the rendered media. Such display components may include, for instance, one or more display screens.

The protective eyewear of some embodiments may include environmental conditioning features that may mitigate or prevent issues such as fogging, user discomfort, etc.

### BRIEF DESCRIPTION OF THE DRAWINGS

The novel features of the disclosure are set forth in the appended claims. However, for purpose of explanation, several embodiments are illustrated in the following drawings.

FIG. 1 illustrates an example overview of one or more embodiments described herein, in which protective eyewear of one or more embodiments provides a sanitized viewing environment;

FIG. 2 illustrates a right-side elevation view of protective eyewear of one or more embodiments described herein;

FIG. 3 illustrates a rear elevation view of protective eyewear of one or more embodiments described herein;

FIG. 4 illustrates a schematic block diagram of protective eyewear of one or more embodiments described herein;

FIG. 5 illustrates a schematic block diagram of an environment of one or more embodiments described herein;

FIG. 6 illustrates a flow chart of an exemplary process that provides user protection via a sanitized viewing environment via the protective eyewear of one or more embodiments described herein;

FIG. 7 illustrates a flow chart of an exemplary process that provides environmental conditioning for the protective eyewear of one or more embodiments described herein;

FIG. 8 illustrates a flow chart of an exemplary process that provides communication via the protective eyewear of one or more embodiments described herein;

FIG. 9 illustrates a flow chart of an exemplary process that allows a user to control operation of the protective eyewear of one or more embodiments described herein; and

FIG. 10 illustrates a schematic block diagram of one or more exemplary devices used to implement various embodiments.

### DETAILED DESCRIPTION OF THE INVENTION

The following detailed description describes currently contemplated modes of carrying out exemplary embodiments. The description is not to be taken in a limiting sense, but is made merely for the purpose of illustrating the general principles of some embodiments, as the scope of the disclosure is best defined by the appended claims.

Various features are described below that can each be used independently of one another or in combination with other features. Broadly, some embodiments generally provide protective eyewear for lasers and other high-intensity sources.

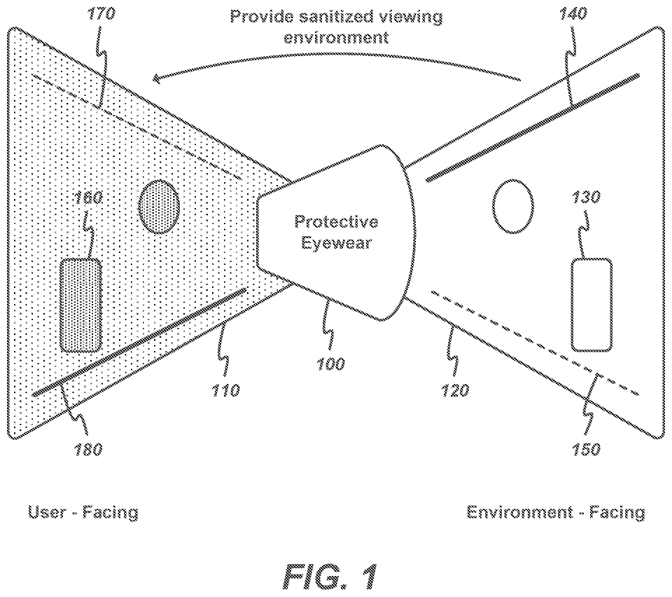

FIG. 1 illustrates an example overview of one or more embodiments described herein, in which protective eyewear 110 of one or more embodiments provides a sanitized viewing environment 110 based on an external viewing environment 120. In this example, the user-facing sanitized viewing environment 110 (e.g., as displayed by the protective eyewear 100) is shown as a mirrored image of the environment-facing external viewing environment 120 (e.g., as captured by the protective eyewear 100), but the user-facing sanitized viewing environment 110 may be presented as the user would perceive the external viewing environment 120, such that, for example, the user may be able to safely move about the physical space associated with the external viewing environment 120.

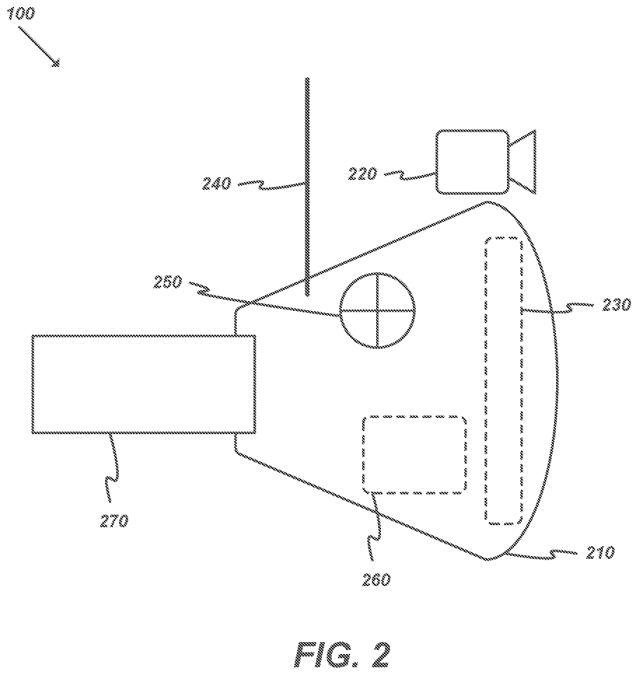

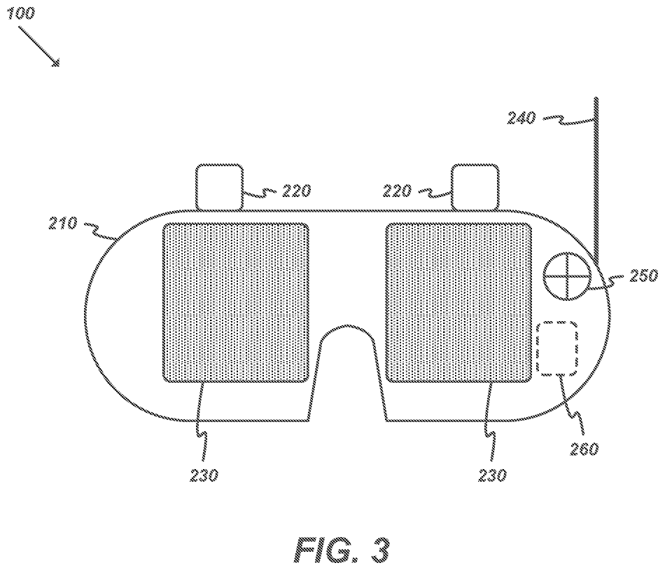

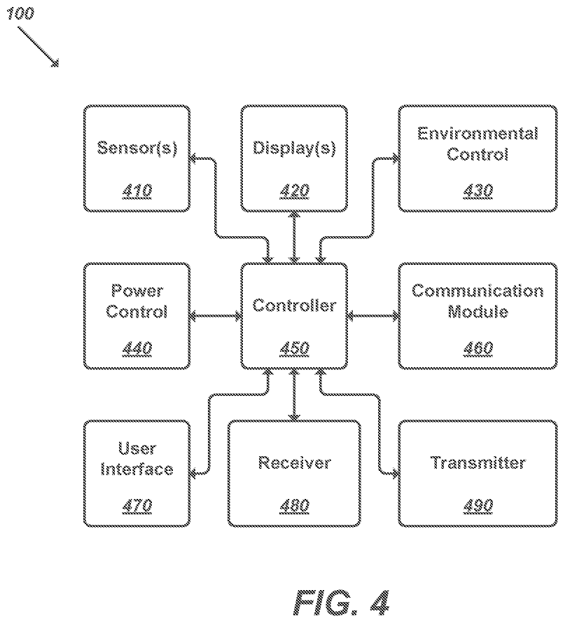

Protective eyewear 100 may include, utilize, and/or otherwise be implemented via one or more electronic devices, components, and/or systems. Protective eyewear 100 may be enclosed in a wearable housing in some embodiments. Protective eyewear 100 will be described in more detail in reference to FIG. 2, FIG. 3, and FIG. 4 below.

Returning to FIG. 1, external viewing environment 120 may be a physical area (e.g., a laboratory such as a laboratory utilizing laser equipment), a virtual reality or augmented reality environment, and/or any other visually perceptible environment that is within a field of view of protective eyewear 100 and/or is otherwise able to be viewed via protective eyewear 100. In some cases, the external viewing environment 120 may be at a different location than the protective eyewear 100, such as when viewing media captured by a drone, a virtual or augmented reality environment, etc.

Objects 130 may include any physical objects that are in the field of view, such as structural elements (e.g., buildings, walls, doors, windows, etc.), tools or other implements, furniture, equipment, people or animals, vehicles, and/or any other visually perceptible objects. In this example, the objects 130 are represented as simple shapes for clarity, but the objects may have any level of detail supported by the protective eyewear 100 (e.g., as defined by camera or display resolution).

Harmful visual element 140 may be an element such as a laser beam or other high-intensity source. Visual elements may be categorized as harmful based on various relevant criteria. For instance, the visual elements may be evaluated to determine various relevant attributes (e.g., intensity, wavelength, etc.) and visual elements with attributes that exceed a specified threshold value may be classified as harmful. In some cases, visual elements may be classified as harmful based on evaluation relative to user preferences or settings. For instance, a first user may indicate that a visual element (e.g., the sun during daytime) is too bright to safely perform tasks under certain conditions and may be classified as harmful, whereas reference to appropriate medical studies indicate that the conditions are marginally acceptable and the visual element may not be classified as harmful.

Non-visible element 150 may include elements that are outside normal human visual perception but that may be perceived by various sensors, cameras, and/or other appropriate components. In this example, the non-visible element 150 is represented as a beam, but non-visible elements 150 may be objects, virtual elements (e.g., tags, metadata, etc.), and/or other information that is not typically perceivable by a user. Examples of non-visible elements 150 may include, for example, infrared light, distant or far-away objects (e.g., signage, equipment, vehicles, etc.), etc.

In some cases, non-visible elements 150 may include elements that are not visible due to attributes of a particular user (e.g., a user with myopia, hyperopia, amblyopia, presbyopia, and/or other visual impairments or conditions). For instance, objects 130 that may be visible to a user with normal visual acuity at a particular distance may not be visible to a myopic user at the same distance. As another example, text that may be readable to a user with normal visual acuity may not be readable to a user with presbyopia. As another example, certain colors may not be able to be differentiated by a user with colorblindness.

In some cases, non-visible content 150 may include elements that are partially obscured and/or obscured for a limited time. For example, if a first user is attempting to view an object across a room and other users are walking in between the first user and the object, the object may be temporarily not visible. As another example, an object may be behind a waving flag, such that all or a portion of the object is not visible at any given time.

In some cases, non-visible content 150 may include data captured via other types of sensors than cameras or visual sensors. For instance, sound information may be captured by a sensor such as a microphone and provided via an element such as representation of non-visible element 180 (e.g., a set of visible waves, speech converted to displayed text, etc.). Other examples include visual representations of sensor data such as temperature, humidity, elevation, location, biometric data (e.g., heart rate of a user), etc.

Sanitized viewing environment 110 may include sanitized objects 160, sanitized visual elements 170, representations of non-visible elements 180, and/or other appropriate content (e.g., user interface (UI) features, text or graphics, indicators, etc.) associated with the external viewing environment 120. The sanitized viewing environment 110 may include visual content that is determined to be non-harmful or non-unwanted. Harmful content may include high-intensity light sources, such as lasers, and/or other elements that are outside some specified acceptance criteria (e.g., an optical power threshold). Unwanted but not necessarily harmful content may include, for example, bothersome or distracting elements such as strobe lights, indicators, elements that obscure a view (e.g., dirt on a window, moving objects, etc.).

In some cases, the sanitized viewing environment 110 may be optimized or otherwise manipulated based on various relevant factors, such as user preferences, analysis of the captured image data, and/or other relevant factors. For instance, the sanitized viewing environment 110 may be provided at different levels of brightness, contrast, zoom, etc. As another example, a user may be able to select a particular point within the external viewing environment 120 that may be used as a focal point for a camera or other sensor. Similarly, such a focal point may be used to adjust attributes of the captured or displayed content (e.g., white balance, color saturation, etc.).

Sanitized objects 160 may provide representations of objects 130 that are determined to be non-harmful and/or otherwise conform to user preferences. For example, a representation of a standard physical object 130 may simply be an image or video stream showing the object.

In some cases, the sanitized objects 160 may be processed or manipulated based on various relevant factors. For instance, if a user is attempting to collect a certain type of compound or material, that compound or material may be represented as an object 160 having a bright color, having a halo effect, and/or some other indicator that the object 160 is associated with the compound or material. As another example, controls for machinery or equipment may be automatically identified and indicated using some visual indicator such as color, graphics, text, etc.

Sanitized visual element 170 may be presented in various different ways, depending on properties of the associated harmful visual content 140, such as type, intensity, etc. For instance, a laser directed at camera of protective eyewear 100 may be removed or obscured in the sanitized viewing environment 110 such that a user may not even be aware of the harmful visual content 140. In some cases, the sanitized visual element 170 may be a representation or indicator associated with the harmful visual content 140. For instance, a laser beam may be represented as a colored line, within safe viewing parameters, that may allow a user to, for example, identify a source of the harmful visual content 140, determine a type of the harmful visual content 140, and/or otherwise evaluate such content.

Representation of non-visible element 180 may include, for example, graphics, text, icons, and/or other representations, as appropriate. In this example, a non-visible element 150 such as a light beam (e.g., an infrared beam) is provided as a representation of non-visible element 180 including a line or beam graphic element.

FIG. 2 illustrates a right-side elevation view of protective eyewear 100 of one or more embodiments described herein. As shown, the protective eyewear 100 may include a housing 210, one or more cameras or other sensors 220, one or more displays 230, a transceiver 240, environmental conditioning elements such as a fan 250, various electronics 260, and a strap or other securing feature 270.

FIG. 3 illustrates a rear elevation view of protective eyewear 100 of one or more embodiments described herein. In this example, the protective eyewear 100 includes two cameras 230 and two displays 230 such that a stereo view is provided and a user may be able to more accurately sense depth.

Although examples above and below may be directed toward protective eyewear 100 with a goggle-type housing, one of ordinary skill in the art will recognize that the protective eyewear 100 of some embodiments may be implemented in various different forms and/or various different ways without departing from the scope of the disclosure. For example, the protective eyewear 100 may be implemented as a component of a helmet, such as a flight helmet. As another example, the protective eyewear 100 may be implemented as eyeglasses.

Housing 210 in this example is a goggle style housing. Housing 210 may include rigid or semi-rigid materials such as plastic, metal, wood, etc. Housing 210 may include various other materials, as appropriate, such as rubber or silicone gaskets or seals where the housing 210 contacts a user. Housing 210 may be opaque and/or include various filtering dyes or other materials that may block all visible and/or harmful visual elements. Housing 210 may surround the eyes of a user (thus creating an “interior” environment of the protective eyewear 100) such that no visual elements are provided to the user except via the display 230. In this example, the “exterior” surfaces of the housing 210 may include those surfaces of the housing 210 that are exposed to the external viewing environment 120 during use and the “interior” surfaces of the housing 210 may include those surfaces that are not exposed to the external viewing environment 120 during use (i.e., those surfaces between the housing and user).

One or more cameras 220 and/or other sensors (e.g., microphones, distance sensors, environmental sensors, biometric sensors, etc.) may capture data related to the external viewing environment 120. The cameras 220 may generally have a high refresh rate to reduce or eliminate lag, and may be able to capture data outside the visible spectrum. In this example, the camera 220 is coupled to the housing 210, but the camera 220 and/or other sensors may be at different locations that are communicatively coupled to the protective eyewear 100. For instance, camera 220 may be associated with a manned or unmanned aircraft or vehicle. As another example, camera 220 may be associated with a fixed location (e.g., a security camera). Camera 220 may be associated with various actuators or other positioning components that may be able to change attributes of the camera 220, such as direction or orientation, location, etc.

Display 230 may include one or more display screens that may be coupled to an interior surface of the protective eyewear 100. Display 230 may include multiple flat and/or curved screens. Display 230 may provide the sanitized viewing environment 110 to a user. The sanitized viewing environment 110 may be provided as rendered media (e.g., images, video, graphics, etc.) that is generated based on data captured by cameras 220 and/or other sensors.

Transceiver 240 may include a transmitter and/or receiver that are able to interact with various other devices, components, systems, etc. For instance, data captured by camera 220 may be sent via transceiver 240 to a remote resource such as a server for storage and/or analysis. As another example, data captured by camera 220 may be sent via transceiver 240 to another instantiation of protective eyewear 100. Likewise, data may be received via transceiver 240 from another instantiation of protective eyewear 100 or resource such as a user device (e.g., a smartphone) or server.

Fan 250 and/or other environmental conditioning features (e.g., a dehumidifier, air conditioner, heater, etc.) may be used to control the operating environment of the protective eyewear 100 and the associated user. For example, the fan 250 may be used to cool the user and to reduce or eliminate fogging of the display 230. Fan 250 may be associated with conduit, shields, and/or other appropriate features that may prevent light from entering the interior environment of protective eyewear 100.

Electronics 260 may include various components and/or circuitry such as processors, interfaces, memory, communication features (e.g., receiver and/or transmitter circuitry), power management or storage (e.g., charging circuitry, one or more batteries, etc.), UI features, image processing components, and/or other appropriate components.

Strap 270 may include various appropriate pliable materials (e.g., elastane, rubber, silicone, etc.). The strap 270 may include various adjustment features, such as clasps, ladder locks, pins, prongs, holes, and/or other features that may allow the protective eyewear 100 to be securely coupled to a user.

One of ordinary skill in the art will recognize that the components of protective eyewear 100 may be arranged in various different ways without departing from the scope of the disclosure. In addition, various components may be omitted from and/or included with the protective eyewear 100 without departing from the scope of the disclosure. For instance, some embodiments of the protective eyewear 100 may not include a transceiver 240. As another example, some embodiments of the protective eyewear 100 may include visual aids such as a flashlight or headlamp. As another example, the protective eyewear 100 may include various UI components (e.g., switches, keypads, buttons, etc.) that may allow a user to at least partly control the operation of the protective eyewear 100.

In this example, the protective eyewear 100 is implemented as a stand-alone device using a single housing 210. However, in some embodiments the various components of protective eyewear 100 may be distributed across multiple housings, devices, and/or other resources. For example, some embodiments of the protective eyewear 100 may include a separate battery pack that is coupled to housing 210 via a cable. As another example, some embodiments of the protective eyewear 100 may include or utilize a camera 220 at a remote location in a separate housing.

FIG. 4 illustrates a schematic block diagram of protective eyewear 100 of one or more embodiments described herein. As shown, the protective eyewear 100 may include sensors 410, displays 420, an environmental control module 430, a power control module 440, a controller 450, a communication module 460, UI module 470, receiver 480, and/or a transmitter 490. One of ordinary skill in the art will recognize that protective eyewear 100 may omit various listed components and/or include various other components, as appropriate.

Sensors 410 may include sensors such as camera 220 described above. Other example sensors include microphones, environmental sensors, biometric sensors, and/or any other appropriate sensors.

Display 420 may be able to provide media to a user. Display 420 may include displays such as display 230 described above. Display 420 may include elements such as display screens or touch screens. Display 420 may include other types of media presentation elements, such as a set of speakers and associated audio circuitry.

Environmental control module 430 may receive information from various sensors 410, such as temperature, humidity, barometric pressure, etc. Such sensors 410 may be associated with the exterior or interior environment of the protective eyewear 100. The environmental control module 430 may at least partly direct the operation of environmental conditioning elements, such as fan 250, based on the received sensor data. For instance, if the interior environment of the protective eyewear 100 exceeds a specified temperature or humidity, the fan 250 may be activated.

Power control module 440 may include components such as charging circuitry and may be associated with various power supply (e.g., a cable or other connected supply), solar cell, and/or storage elements such as batteries. Power control module 440 may interact with other modules, such as the environmental control module 430, UI module 470, and/or other components of the protective eyewear 100 in order to limit power consumed by the protective eyewear 100 and thus extend battery life, as appropriate for applications such as using the protective eyewear 100 in a laboratory setting for several hours.

Controller 450 may be, include, and/or utilize various electronic components and/or circuitry that are able to execute instructions and/or otherwise process data. Controller 450 may at least partly direct the operations of the other components of protective eyewear 100.

Communication module 460 may include various interfaces and/or implement various messaging protocols that may allow the protective eyewear 100 to communicate with other components, devices, and/or systems across various channels. For instance, communication module 460 may utilize radio frequency (RF) transmitters 490 and/or receivers 480. As another example, communication module 460 may be able to facilitate communication using various network interfaces (e.g., Ethernet, Wi-Fi, cellular networks, the Internet, etc.).

UI module 470 may include various components and/or interfaces that may allow the protective eyewear 100 to interact with various UI elements (not shown). For instance, protective eyewear 100 may include a keypad, buttons, knobs, touchscreens, and/or other UI elements (e.g., haptic feedback elements, audio outputs, display screens, etc.) that may be utilized by UI module 470. In some embodiments, UI module 470 may provide various graphical UI (GUI) features, such as text, icons or other graphics, etc. provided via a UI feature such as a touchscreen. UI module 470 may receive user inputs and provide the received data to resources such as controller 450.

Environmental control module 430, power control module 440, controller 450, communication module 360, UI module 470, and/or other components of the protective eyewear 100 may be, be included in, be associated with, and/or otherwise be utilized by the electronics 260 described above.

Receiver 480 and/or transmitter 490 may allow protective eyewear 100 to communicate across various channels, such as RF channels, optical channels, etc. In some embodiments, receiver 480 and transmitter 490 may be implemented at different locations and/or otherwise associated with different components, devices, and/or systems. For instance, a protective eyewear 100 associated with a remote camera 220 may include a transmitter 490 with the remote camera 220. Signals from that transmitter may be sent to a receiver 480 from a second set of protective eyewear 100 such that captured image data may be sent to and displayed at the second set of protective eyewear 100. Receiver 480 and/or transmitter 490 may be included in, be associated with, and/or otherwise be utilized by transceiver 240 described above.

One of ordinary skill in the art will recognize that protective eyewear 100 may include various other components than shown, and/or omit various listed components. For instance, protective eyewear 100 may include memory for storing instructions and/or data. As another example, some embodiments of the protective eyewear 100 may save power and extend battery life by omitting the receiver 480 and/or transmitter 490. As another example, some embodiments of the protective eyewear 100 may include multiple receivers 480 and/or multiple transmitters 490 for communicating across different channels or types of channels.

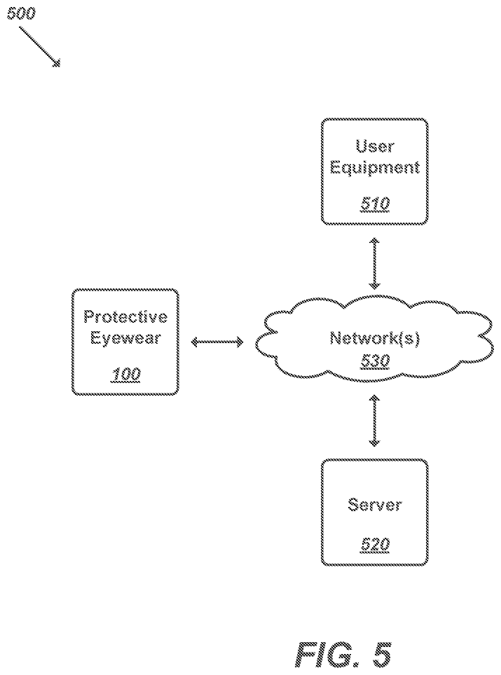

FIG. 5 illustrates a schematic block diagram of an environment 500 of one or more embodiments described herein. As shown, the environment 500 may include one or more protective eyewear 100, user equipment 510, servers 520, and/or more networks 530.

User equipment 510 may be an electronic device such as a smartphone, tablet, personal computer, wearable device, and/or other appropriate devices. User equipment 510 may include user interface components (e.g., a touchscreen, displays, buttons, keypads, etc.) that may be utilized in conjunction with protective eyewear 100.

Server 520 may be, include, and/or utilize a set of electronic components that are able to execute instructions, process data, receive data, store data, and/or provide stored data. Some embodiments may utilize multiple types of servers 520 and/or associated storages, and/or some servers 520 may perform multiple types of functions. For instance, a server 520 may act as a media server that stores captured and/or sanitized media and/or provides such media to other resources. As another example, server 520 may store profile information related to each instantiation of protective eyewear 100, users, operating environments, and/or other relevant entities. As another example, information such as firmware updates may be provided via server 520. As another example, protective eyewear 100 may connect to a third-party resource such as a server 520 that provides weather information, in order to retrieve weather data related to a particular operating environment. Server 520 and/or other such resources may be associated with one or more storages that may be accessible via a resource such as an application programming interface (API).

Network(s) 530 may include wired networks (e.g., Ethernet), wireless networks (e.g., Wi-Fi, cellular networks, the Internet, etc.), wired or wireless communication channels (e.g., universal serial bus (USB), Bluetooth, etc.), and/or other appropriate communication channels.

Environment 500 may include multiple instantiations of protective eyewear 100 that may be able to communicate across networks 530 in order to share captured media, provide communication pathways, and/or otherwise allow interaction among the protective eyewear 100 and/or users thereof.

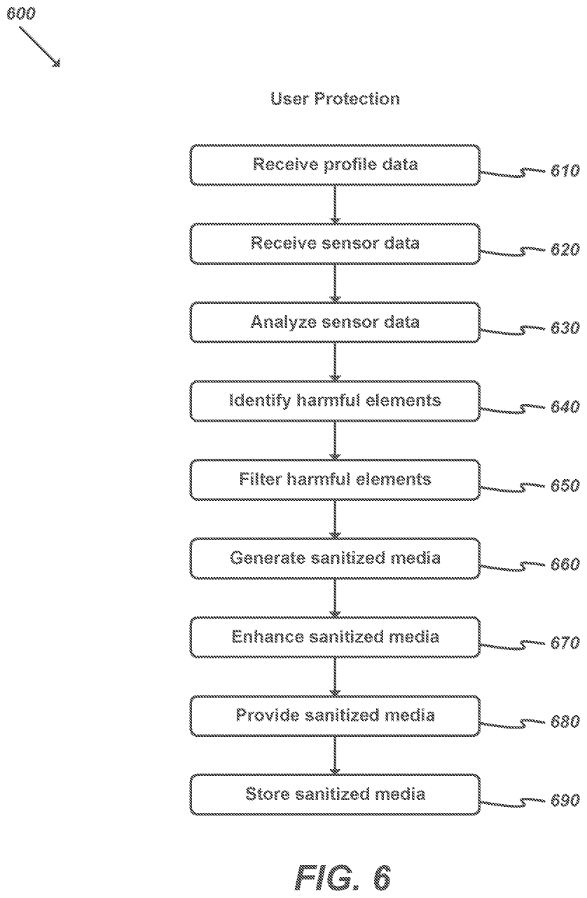

FIG. 6 illustrates an example process 600 for providing user protection via a sanitized viewing environment via the protective eyewear 100. Such a process may allow users to work in dangerous or harmful visual environments without being exposed to harmful visual content. The process may be performed, for example, when the protective eyewear 100 of some embodiments is powered on. In some embodiments, process 600 may be performed by protective eyewear 100.

The process may include receiving (at 610) profile data. Profile data may be received from a resource such as a server 520. Profile data may include, for example, user profile data, protective eyewear 100 device data, environment profile data, and/or other such relevant data. Each entity (e.g., each user, each instantiation of protective eyewear 100, and/or other appropriate elements) may be associated with a unique identifier such as a username or serial number. Profile data may be stored using a resource such as a lookup table or other database. The unique identifiers may be utilized to determine which entry in such a lookup table is associated with a particular entity.

Different types of profiles may include various different types of information. For instance, a user profile may include information such as user preferences, visual impairments, user type, and/or other relevant information. As another example, a profile associated with an instantiation of protective eyewear 100 may include information such as type (e.g., goggle, helmet, etc.), associated sensors or other components (e.g., a listing of available cameras and/or other sensors), communication capabilities, a listing of user identifiers associated with authorized users for the protective eyewear 100, and/or other appropriate information. Profiles may be associated with components such as sensors 410. Such a sensor profile may include information such as sensor type, output type or range, calibration data, and/or other relevant information. Of particular importance will be profile data that identifies and describes potential hazards that may be encountered during the course of operations.

In some cases, profile data may be automatically retrieved based on some available information. For instance, an instantiation of protective eyewear 100 may be associated with a unique ID that may be transmitter to a resource such as server 520. The server 520 may identify a profile associated with the protective eyewear 100 and retrieve the associated profile. The protective eyewear profile may include information such as a user identifier for an associated user. The user identifier may be used to retrieve the user profile information. In other cases, a user may have to log in using, for example, a username and/or password for the appropriate profile to be received. In some cases, a default profile may be utilized (e.g., a default user profile may be used when the user cannot be identified or has not previously stored a profile).

As shown, process 600 may include receiving (at 620) sensor data. Sensor data may be received from resources such as camera 220 and/or other sensors 410. Sensor data may be received at regular intervals (e.g., frames of video may be captured at an appropriate frame rate, data from environmental sensors may be received periodically, etc.) and/or based on some relevant criteria.

Process 600 may include analyzing (at 630) the received sensor data. Such analysis may include, for instance, quantification of various visual attributes (e.g., light intensity), comparison of measured values to one or more thresholds, and/or other appropriate analysis. Such comparison may be performed on a pixel-by-pixel basis in some embodiments (e.g., each pixel that exceeds a light intensity threshold may be identified and added to a listing of pixels denoting potentially harmful sources). Some embodiments of the protective eyewear 100 may utilize machine learning modules or other artificial intelligence to analyze the received data.

The process may include identifying (at 640) harmful elements based on the analysis. Thus, for instance, visual elements that exceed a specified light intensity threshold may be identified. Such visual elements may be defined in various appropriate ways, and/or including various relevant data. For example, elements identified as harmful or unsafe may be defined by determining the associated frame(s) of video, location of the element within the frame (e.g., by specifying a set of pixels that define a range or shape), and/or other relevant attributes.

As shown, process 600 may include filtering (at 650) harmful elements. Elements may be filtered in different ways depending on the element attributes, user profile information, capabilities of the protective eyewear 100 and/or other relevant factors. For instance, visual elements that are determined to be harmful or unsafe (e.g., those that exceed a maximum light intensity) may be rendered harmless from the user view in the sanitized viewing environment 110 when the visual elements saturate pixels in the capturing camera (e.g., camera 220). Such saturated pixels may be given a unique display pattern, such as blinking, to let the user know of their existence and location. Further filtering may be performed based on identification of rapid changes in source position, user preferences, and/or other relevant factors.

Process 600 may include generating (at 660) sanitized media. Generating the sanitized media may include using the filtered sensor data (e.g., captured video with harmful visual elements dimmed or removed) to render the sanitized media. As described above, the sanitized media may include sanitized objects 160, sanitized visual elements 170, and/or other similar elements.

The process may include enhancing (at 670) the sanitized media. Enhancement may be performed based on various relevant factors, such as user preference, visual impairments of the user, presence of non-visible elements 150, and/or other relevant factors. In some cases, the sanitized media content may be enhanced before rendering the sanitized media.

As shown, process 600 may include providing (at 680) the sanitized media. The sanitized media may be provided via a resource such as display 230 and/or other appropriate resources.

Process 600 may include storing (at 690) the sanitized media. The sanitized media may be stored at the protective eyewear 100, or sent to a resource such as a media server 520 for storage. Stored media may be analyzed to review performance, update operating procedures, evaluate exposure to harmful conditions, and/or otherwise utilized as appropriate.

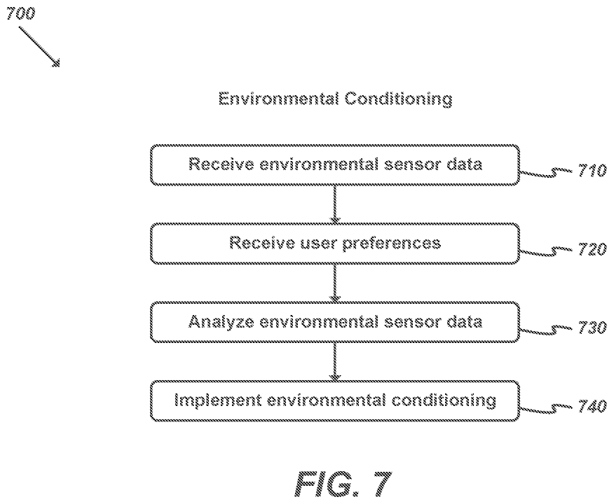

FIG. 7 illustrates an example process 700 for providing environmental conditioning for the protective eyewear 100. Such environment conditioning may be utilized to improve user comfort, reduce fogging of displays, and/or otherwise provide a suitable usage environment. The process may be performed when the protective eyewear 100 is powered on. In some embodiments, process 700 may be performed by protective eyewear 100.

As shown, process 700 may include receiving (at 710) environmental sensor data. Sensor data may be received in a similar manner to that described above. Sensor data may include, for instance, interior and/or exterior sensor data including temperature, humidity, etc. In addition, profile information (e.g., user profile information, protective eyewear 100 profile information, sensor profile information, etc.) may be received from a resource such as server 520.

The process may include receiving (at 720) user preferences. For instance, a user may set a range of desired operating temperatures or humidity range that may be stored with the user profile.

Process 700 may include analyzing (at 730) the received environmental sensor data. Such data may be analyzed, for example, by comparing the received sensor information to various thresholds associated with the various profiles. As another example, each type of protective eyewear 100 may be associated with a profile that indicates threshold values for the various types of environmental sensor data.

As shown, process 700 may include implementing (at 740) environmental conditioning. The environmental conditioning may depend on the analysis of sensor data, available conditioning resources, and/or other relevant factors. For instance, if an interior temperature exceeds a specified threshold, a resource such as fan 250 may be activated.

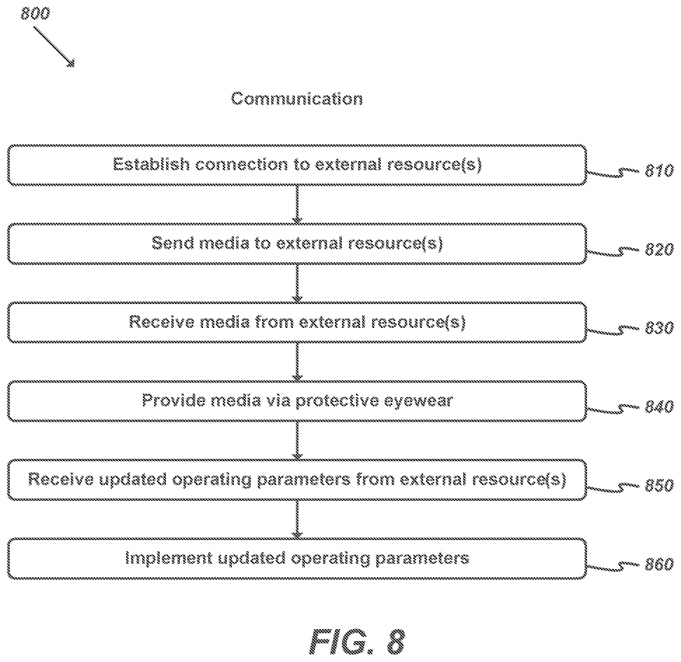

FIG. 8 illustrates an example process 800 for providing communication via the protective eyewear 100. Such a process may allow data to be shared among multiple devices, components, and/or systems, such as multiple instantiations of the protective eyewear 100. The process may be performed when a connection to an external resource is requested, when a request to connect is received, and/or under other appropriate conditions. In some embodiments, process 800 may be performed by protective eyewear 100.

As shown, process 800 may include establishing (at 810) a connection to one or more external resources. Such a connection may be established across various local and/or distributed elements, such as via networks 530. Such a connection may be established using various sets of messages and/or other appropriate ways.

Process 800 may include sending (at 820) media to the external resources. Such media may include, for example, video data captured by camera 220, sanitized media rendered by the protective eyewear 100, and/or other media. The media may be sent to external resources such as a server 520, user equipment 510, and/or other instantiations of the protective eyewear 100. Media may be sent via a resource such as networks 530.

The process may include receiving (at 830) media from the external resources. Such media may include, for example, video data captured by other instantiations of the protective eyewear 100, sanitized media rendered by other instantiations of the protective eyewear 100, and/or other media (e.g., media stored at server 520). The media may be received from external resources such as server 520, user equipment 510, and/or other instantiations of the protective eyewear 100. Media may be received via a resource such as networks 530. In some cases, media may be received from remote components of the protective eyewear 100, such as a camera 220 associated with a drone or remote location.

As shown, process 800 may include providing (at 840) media via the protective eyewear 100. Media may be provided via resources such as display 230. In this way, a first user may be able to share sanitized media, such that a second user may be able to provide additional evaluation and/or oversight. As another example, multiple users may be able to monitor media captured by a remote camera 220 or other sensor.

Process 800 may include receiving (at 850) updated operating parameters from the external resources. For example, firmware or other operating software updates may be received at the protective eyewear 100 from a resource such as a server 520. As another example, machine learning models, evaluation thresholds, and/or other information related to evaluation of visual events may be distributed from a resource such as server 520 to each instantiation of the protective eyewear 100.

The process may include implementing (at 860) the updated operating parameters. For example, the received firmware or evaluation threshold data may be applied at the protective eyewear 100 by replacing previously stored data, by installing or compiling software, and/or otherwise implementing the updated operating parameters.

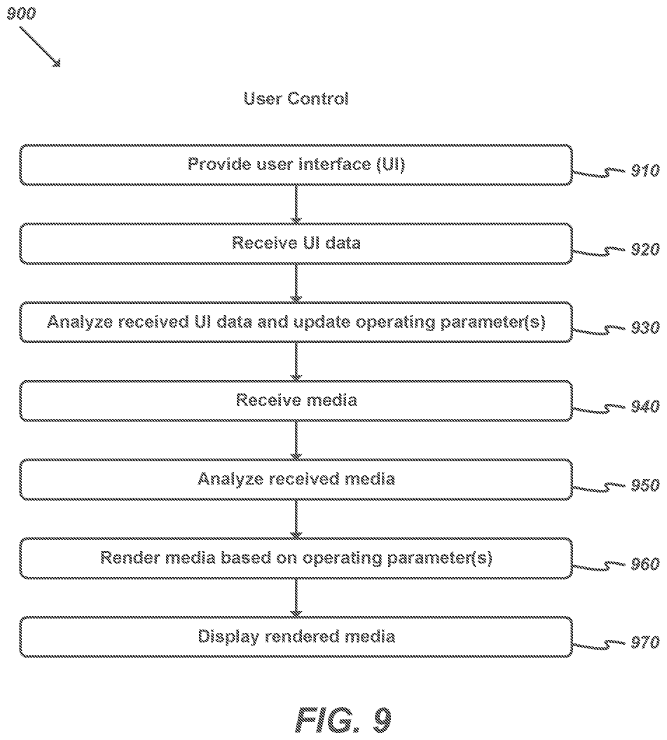

FIG. 9 illustrates an example process 900 for allowing a user to control operation of the protective eyewear 100. Such a process may allow a user to adjust viewing parameters (e.g., brightness, contrast, etc.) or attributes, define unwanted visual events, set thresholds, and/or otherwise control the protective eyewear 100. The process may be performed when media is provided via the protective eyewear 100, such as via display 230. In some embodiments, process 900 may be performed by protective eyewear 100.

As shown, process 900 may include providing (at 910) a UI. Such a UI may be provided via various components of the protective eyewear 100, such as display 230, buttons, knobs, keypads, and/or other such features associated with housing 110, and/or via another resource such as user equipment 510.

Process 900 may include receiving (at 920) UI data. The UI data may be received from the various components and/or GUI elements. For instance, if UI data indicates a “brightness” knob has been rotated clockwise, a brightness parameter may be determined based on the final position of the knob. As another example, user temperature preferences may be received via a GUI provided via user equipment 510.

The process may include analyzing (at 930) the received UI data and updating operating parameters. The received UI data may be analyzed and compared to current settings to identify updates to the operating parameters.

As shown, process 900 may include receiving (at 940) media. Such media may be received locally (e.g., media generated at protective eyewear 100, media captured by camera 220, etc.) and/or from external resources such as user device 510, a media server 520, and/or other instantiations of the protective eyewear 100.

Process 900 may include analyzing (at 950) the received media. Such analysis may include, for example, determining various relevant attributes of the received media and comparing such attributes to the operating parameter thresholds associated with the received UI data.

Process 900 may include rendering (at 960) media based on the operating parameter(s). Such rendering may include, for example, increasing or decreasing brightness and/or other attributes associated with the received UI data.

The process may include displaying (at 970) the rendered media. The rendered media may be displayed via a resource such as display 230.

Process 900, or portions thereof, may be performed iteratively, such that a user may update operating parameters during use.

One of ordinary skill in the art will recognize that processes 600-900 may be implemented in various different ways without departing from the scope of the disclosure. For instance, the elements may be implemented in a different order than shown. As another example, some embodiments may include additional elements or omit various listed elements. Elements or sets of elements may be performed iteratively and/or based on satisfaction of some performance criteria. Non-dependent elements may be performed in parallel. Elements or sets of elements may be performed continuously and/or at regular intervals.

The processes and modules described above may be at least partially implemented as software processes that may be specified as one or more sets of instructions recorded on a non-transitory storage medium. These instructions may be executed by one or more computational element(s) (e.g., microprocessors, microcontrollers, digital signal processors (DSPs), application-specific integrated circuits (ASICs), field programmable gate arrays (FPGAs), other processors, etc.) that may be included in various appropriate devices in order to perform actions specified by the instructions.

As used herein, the terms “computer-readable medium” and “non-transitory storage medium” are entirely restricted to tangible, physical objects that store information in a form that is readable by electronic devices.

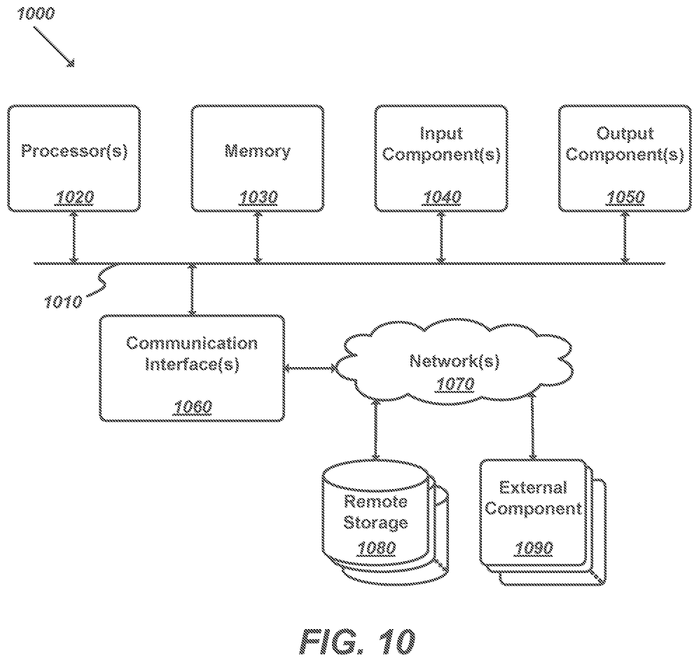

FIG. 10 illustrates a schematic block diagram of an exemplary device (or system, or devices, or components) 1000 used to implement some embodiments. For example, the systems, devices, components, and/or operations described above in reference to FIG. 1, FIG. 2, FIG. 3, FIG. 4, and FIG. 5 may be at least partially implemented using device 1000. As another example, the processes described in reference to FIG. 6, FIG. 7, FIG. 8, and FIG. 9 may be at least partially implemented using device 1000.

Device 1000 may be implemented using various appropriate elements and/or sub-devices. For instance, device 1000 may be implemented using one or more personal computers (PCs), servers, mobile devices (e.g., smartphones), tablet devices, wearable devices, and/or any other appropriate devices. The various devices may work alone (e.g., device 1000 may be implemented as a single smartphone) or in conjunction (e.g., some components of the device 1000 may be provided by a mobile device while other components are provided by a server).

As shown, device 1000 may include at least one communication bus 1010, one or more processors 1020, memory 1030, input components 1040, output components 1050, and one or more communication interfaces 1060.

Bus 1010 may include various communication pathways that allow communication among the components of device 1000. Processor 1020 may include a processor, microprocessor, microcontroller, DSP, logic circuitry, and/or other appropriate processing components that may be able to interpret and execute instructions and/or otherwise manipulate data. Memory 1030 may include dynamic and/or non-volatile memory structures and/or devices that may store data and/or instructions for use by other components of device 1000. Such a memory device 1030 may include space within a single physical memory device or spread across multiple physical memory devices.

Input components 1040 may include elements that allow a user to communicate information to the computer system and/or manipulate various operations of the system. The input components may include keyboards, cursor control devices, audio input devices and/or video input devices, touchscreens, motion sensors, etc. Output components 1050 may include displays, touchscreens, audio elements such as speakers, indicators such as light-emitting diodes (LEDs), printers, haptic or other sensory elements, etc. Some or all of the input and/or output components may be wirelessly or optically connected to the device 1000.

Device 1000 may include one or more communication interfaces 1060 that are able to connect to one or more networks 1070 or other communication pathways. For example, device 1000 may be coupled to a web server on the Internet such that a web browser executing on device 1000 may interact with the web server as a user interacts with an interface that operates in the web browser. Device 1000 may be able to access one or more remote storages 1080 and one or more external components 1090 through the communication interface 1060 and network 1070. The communication interface(s) 1060 may include one or more APIs that may allow the device 1000 to access remote systems and/or storages and also may allow remote systems and/or storages to access device 1000 (or elements thereof).

It should be recognized by one of ordinary skill in the art that any or all of the components of computer system 1000 may be used in conjunction with some embodiments. Moreover, one of ordinary skill in the art will appreciate that many other system configurations may also be used in conjunction with some embodiments or components of some embodiments.

In addition, while the examples shown may illustrate many individual modules as separate elements, one of ordinary skill in the art would recognize that these modules may be combined into a single functional block or element. One of ordinary skill in the art would also recognize that a single module may be divided into multiple modules.

Device 1000 may perform various operations in response to processor 1020 executing software instructions stored in a computer-readable medium, such as memory 1030. Such operations may include manipulations of the output components 1050 (e.g., display of information, haptic feedback, audio outputs, etc.), communication interface 1060 (e.g., establishing a communication channel with another device or component, sending and/or receiving sets of messages, etc.), and/or other components of device 1000.

The software instructions may be read into memory 1030 from another computer-readable medium or from another device. The software instructions stored in memory 1030 may cause processor 1020 to perform processes described herein. Alternatively, hardwired circuitry and/or dedicated components (e.g., logic circuitry, ASICs, FPGAs, etc.) may be used in place of or in combination with software instructions to implement processes described herein. Thus, implementations described herein are not limited to any specific combination of hardware circuitry and software.

The actual software code or specialized control hardware used to implement an embodiment is not limiting of the embodiment. Thus, the operation and behavior of the embodiment has been described without reference to the specific software code, it being understood that software and control hardware may be implemented based on the description herein.

While certain connections or devices are shown, in practice additional, fewer, or different connections or devices may be used. Furthermore, while various devices and networks are shown separately, in practice the functionality of multiple devices may be provided by a single device or the functionality of one device may be provided by multiple devices. In addition, multiple instantiations of the illustrated networks may be included in a single network, or a particular network may include multiple networks. While some devices are shown as communicating with a network, some such devices may be incorporated, in whole or in part, as a part of the network.

Some implementations are described herein in conjunction with thresholds. To the extent that the term “greater than” (or similar terms) is used herein to describe a relationship of a value to a threshold, it is to be understood that the term “greater than or equal to” (or similar terms) could be similarly contemplated, even if not explicitly stated. Similarly, to the extent that the term “less than” (or similar terms) is used herein to describe a relationship of a value to a threshold, it is to be understood that the term “less than or equal to” (or similar terms) could be similarly contemplated, even if not explicitly stated. Further, the term “satisfying,” when used in relation to a threshold, may refer to “being greater than a threshold,” “being greater than or equal to a threshold,” “being less than a threshold,” “being less than or equal to a threshold,” or other similar terms, depending on the appropriate context.

No element, act, or instruction used in the present application should be construed as critical or essential unless explicitly described as such. An instance of the use of the term “and,” as used herein, does not necessarily preclude the interpretation that the phrase “and/or” was intended in that instance. Similarly, an instance of the use of the term “or,” as used herein, does not necessarily preclude the interpretation that the phrase “and/or” was intended in that instance. Also, as used herein, the article “a” is intended to include one or more items and may be used interchangeably with the phrase “one or more.” Where only one item is intended, the terms “one,” “single,” “only,” or similar language is used. Further, the phrase “based on” is intended to mean “based, at least in part, on” unless explicitly stated otherwise.

The foregoing relates to illustrative details of exemplary embodiments and modifications may be made without departing from the scope of the disclosure. Even though particular combinations of features are recited in the claims and/or disclosed in the specification, these combinations are not intended to limit the possible implementations of the disclosure. In fact, many of these features may be combined in ways not specifically recited in the claims and/or disclosed in the specification. For instance, although each dependent claim listed below may directly depend on only one other claim, the disclosure of the possible implementations includes each dependent claim in combination with every other claim in the claim set.

We claim:

1. A device, comprising:
one or more processors configured to:
capture, at a protective eyewear device, visual data associated with an operating environment of the protective eyewear device;
analyze the visual data in order to identify harmful visual elements;
filter the harmful visual elements from the visual data to generate filtered visual data;
render sanitized media based on the filtered visual data; and
display the sanitized media via a display of the protective eyewear device.

2. The device of claim 1, wherein harmful visual events are identified by determining whether a light intensity of each pixel of the visual data exceeds a specified threshold.

3. The device of claim 2, the one or more processors further configured to compile a listing of harmful visual events including a listing of pixels associated with each harmful visual event.

4. The device of claim 1, wherein the protective eyewear device comprises an opaque housing and a camera coupled to an exterior surface of the opaque housing, and wherein the visual data is captured via the camera.

5. The device of claim 4, wherein the display comprises a display screen coupled to an interior surface of the opaque housing.

6. The device of claim 1, the one or more processors further configured to store the sanitized media at the protective eyewear device.

7. The device of claim 1, the one or more processors further configured to send the captured visual data and sanitized media to a media server.

8. A non-transitory computer-readable medium, storing a plurality of processor-executable instructions to:
capture, at a protective eyewear device, visual data associated with an operating environment of the protective eyewear device;
analyze the visual data in order to identify harmful visual elements;
filter the harmful visual elements from the visual data to generate filtered visual data;
render sanitized media based on the filtered visual data; and
display the sanitized media via a display of the protective eyewear device.

9. The non-transitory computer-readable medium of claim 8, wherein harmful visual events are identified by determining whether a light intensity of each pixel of the visual data exceeds a specified threshold.

10. The non-transitory computer-readable medium of claim 9, the plurality of processor-executable instructions further to compile a listing of harmful visual events including a listing of pixels associated with each harmful visual event.

11. The non-transitory computer-readable medium of claim 8, wherein the protective eyewear device comprises an opaque housing and a camera coupled to an exterior surface of the opaque housing, and wherein the visual data is captured via the camera.

12. The non-transitory computer-readable medium of claim 11, wherein the display comprises a display screen coupled to an interior surface of the opaque housing.

13. The non-transitory computer-readable medium of claim 8, the plurality of processor-executable instructions further to store the sanitized media at the protective eyewear device.

14. The non-transitory computer-readable medium of claim 8, the plurality of processor-executable instructions further to send the captured visual data and sanitized media to a media server.

15. A method comprising
capturing, at a protective eyewear device, visual data associated with an operating environment of the protective eyewear device;
analyzing the visual data in order to identify harmful visual elements;
filtering the harmful visual elements from the visual data to generate filtered visual data;
rendering sanitized media based on the filtered visual data; and
displaying the sanitized media via a display of the protective eyewear device.

16. The method of claim 15, wherein harmful visual events are identified by determining whether a light intensity of each pixel of the visual data exceeds a specified threshold.

17. The method of claim 16 further comprising compiling a listing of harmful visual events including a listing of pixels associated with each harmful visual event.

18. The method of claim 15, wherein the protective eyewear device comprises an opaque housing and a camera coupled to an exterior surface of the opaque housing, and wherein the visual data is captured via the camera.

19. The method of claim 18, wherein the display comprises a display screen coupled to an interior surface of the opaque housing.

20. The method of claim 15 further comprising:
storing the sanitized media at the protective eyewear device; and
sending the captured visual data and sanitized media to a media server.
